(19) United States
(12) Patent Application Publication
(10) Pub. No.: US 2007/0134315 A1
(43) Pub. Date: Jun. 14, 2007

(54) Orally administrable extended release pellet and tablet formulations of a highly water soluble compound

(76) Inventors: Michael L. Viera, Gaithersburg, MD (US); Padmanabh P. Bhatt, Rockville, MD (US); Lisa McKnight, Fairfax, VA (US); Abraham Bashai Woldu, North Wales, PA (US); Goutam Muhuri, Piscataway, NJ (US)

Correspondence Address: FOLEY AND LARDNER LLP;SUITE 500, 3000 K STREET NW, WASHINGTON, DC, 20007, US

(21) Appl. No.: 11/296,212

(22) Filed: Dec. 8, 2005

(51) Int. Cl. A61K 9/48; A61K 9/26; A61K 31/165

(52) U.S. Cl. 424/451; 424/470; 514/617

(57) ABSTRACT

Pharmaceutical compositions comprising an extended release formulation of active compounds effective in the treatment of various pathological conditions are provided. More particularly, the invention provides methods of making and using extended release formulations comprising active compounds that present formulation challenges such as short biological half-life, instability, highly water soluble and/or high dose requirements. Specifically, orally administrable extended release pellet and tablet formulations of isovaleramide are preferred.

### FIELD OF THE INVENTION

This invention provides the preparation and use of pharmaceutical compositions of active compounds effective in treatment of various pathological conditions. More particularly, the invention provides methods of making and using extended release formulations comprising active compounds that present formulation challenges such as short biological half-life, instability, high water soluble and/or high dose requirements.

### BACKGROUND OF THE INVENTION

A variety of pathological conditions are presently being treated with active compounds that can be administered orally. In order to be orally administrable, the active pharmaceutical ingredient (API) is formulated with certain excipients that permit the API release inside the body at the desired rate. It has been discovered that certain active compounds have a short biological half-life in humans. In the absence of an approach to reduce the rate of clearance of drug following administration, the short biological half-life requires that it be frequently administered to sustain a therapeutic concentration without adverse effects. Multiple dosing of a conventional immediate release dosage form may exhibit undesired large peak to trough differentials that in turn can be associated with unwarranted side effects or loss of therapeutic control. Furthermore, multiple daily dosing regimens, due to poor patient compliance, are susceptible to skipped doses that again produce fluctuations in drug plasma levels. These situations are critical in the treatment of many pathological conditions such as CNS disorders where it is necessary to maintain steady state drug plasma levels within a specified therapeutic window. Thus, it would be desirable to have a formulation that would permit such an extended release of the API so that the drug can be administered on a once or twice per day schedule.

It has also been discovered that certain API exhibit temperature dependent instability. A rise in the temperature beyond a certain threshold during the formulation process can lead to degradation, physical incompatibilities such as precipitation, sublimation, decomposition, and other such physical as well as chemical changes. Thus, the preparation of an extended release formulation of such active compounds can pose a challenge based on currently available methods.

It has also been discovered that certain active compounds exhibit high solubility in common solvents used in preparing formulations such as water and organic solvents. Highly water-soluble compounds present the problem in that traditional formulation systems do not effectively control the rate of release of such compounds in vivo. Moreover, compounds that exhibit high solubility in the solvents used to process pharmaceutical compositions, such as alcohols and water, present challenges when these solvents are used in processes in the manufacture of extended release formulations. For example, processing challenges arise when polymers, employed to control the rate of drug release, are applied to a drug-containing formulation using a solvent system. In this case, the highly soluble compound can be solubilized in the polymer solvent system and becomes embedded in the polymer film as the solvent evaporates thus imparting an undesired altering of the rate controlling properties of the polymer. Moreover, for compounds that have a relatively short biological half-life and are highly water soluble, the challenges of preparing a formulation are magnified since the API gets rapidly cleared from the biosystem. One way of overcoming this hurdle is by administering multiple high doses of the API to the patient. However, this poses a challenge due to patient compliance issues. Thus, it would be highly desirable to have an orally administrable extended release formulation that can overcome these problems and lead to effective therapy as well as patient compliance.

### SUMMARY OF THE INVENTION

Thus, the present invention provides extended release pharmaceutical compositions of active compounds that can be dosed up to twice daily to meet the steady state plasma levels required for the treatment of pathological conditions. Conditions that would benefit from this invention include CNS disorders such as epilepsy, migraine, bipolar disorder, spasticity and neuropathic pain. The compositions can be tailored to provide various release rates such that multiple options are provided for in vivo drug release times such as 6 hours, 8 hours, 12 hours, 16 hours, 20 hours, 24 hours and the like.

In accomplishing these and other objectives, there has been provided, according to one aspect of the present invention, a pharmaceutical composition comprising an extended release formulation of active compounds such as isovaleramide and other active compounds that are highly water soluble. Upon oral administration, the extended release formulation releases the dose of the active compound over a period of 8 to 24 hours (h). With such a formulation, only one or two administrations of the drug need to be given each day.

In accordance with another embodiment of the present invention, there has been provided, a pharmaceutical composition comprising an extended release formulation of active compounds that exhibit temperature dependent instability such as sublimation, decomposition or precipitation at elevated temperature. According to this invention, the phrase “elevated temperature” refers to a temperature range of approximately 30° C. to approximately 55° C.

In accordance with another embodiment of the present invention, the extended release formulation comprises a unitary body comprising the API that can be easily administered orally. In one embodiment, the unitary body is a capsule comprising multiparticulate pellets. In another embodiment, the unitary body is a tablet.

In accordance with another embodiment of the present invention, each pellet in the capsule comprises an inner core coated with a substantially uniform layer of a rate controlling polymer system. The inner core comprises the API along with two or more excipients such as fillers and binding agents that impart certain characteristic properties to the inner core.

In accordance with yet another embodiment of the present invention, the characteristic properties of the inner core include non-friability during the processing of the inner core such as drying, storage, coating and encapsulation.

In accordance with still another embodiment of the present invention, the rate controlling polymer system retards the access of liquids to the inner core and/or retards the release of the API from the inner core. In one embodiment of the present invention, the rate limiting polymer system comprises polymers of ammonio methacrylate copolymer, cellulose derivatives, polyvinyl acetate or any copolymers and derivatives thereof.

In accordance with another embodiment of the present invention, the inner core is optionally coated with a substantially uniform layer of a sealing coat between the inner core and the rate controlling polymer system. In one embodiment of the present invention, the sealing coat comprises the polymer, hypromellose.

In accordance with one embodiment of the present invention, each tablet comprises the API optionally mixed with excipients such as hydrophilic or hydrophobic rate controlling polymers, hydrophobic matrix forming compounds, binders, fillers and bulking agents.

In accordance with still another embodiment of the present invention, the rate controlling polymers retard the access of liquids into the tablet and/or retard the release of the API from the tablet. In one embodiment of the present invention, the rate controlling polymers comprise polymers of ammonio methacrylate, cellulose derivatives, poly (ethylene oxide), carbomer, gums, polyvinyl acetate or any copolymers and derivatives thereof.

In accordance with another embodiment of the present invention, the tablet is optionally coated with a substantially uniform layer of film coating to make it more suitable for oral administration.

In accordance with another aspect of the present invention, a process is provided for producing the extended release formulation described above.

In accordance with another aspect of the present invention, a method of treating pathological disorders such as convulsions, bipolar affective disorder, migraine, anxiety, and other CNS disorders comprising oral administration of the extended release formulation is described.

Other objects, features, and advantages of the present invention will become apparent from the following detailed description. It should be understood, however, that the detailed description and the specific examples, while indicating preferred embodiments of the present invention, are given by way of illustration only, since various changes and modifications within the spirit and scope of the invention will become apparent to those skilled in the art from the detailed description.

### BRIEF DESCRIPTION OF THE DRAWINGS

FIG. 1 depicts plots of the isothermal thermogravimetric analysis (TGA) results for isovaleramide.

FIG. 2 shows the dissolution profile for an encapsulated isovaleramide immediate release pellet formulation using the USP Apparatus II at 75 RPM and buffered media, pH 6.8 (±0.1) at 37° C. (±0.5° C.).

FIG. 3 shows the dissolution profile for encapsulated isovaleramide extended release pellet compositions using the USP Apparatus II at 75 RPM and buffered media, pH 6.8 (±0.1) at 37° C. (±0.5° C.).

FIG. 4 shows the dissolution profile for isovaleramide extended release tablet compositions using the USP Apparatus II at 75 RPM and buffered media, pH 6.8 (±0.1) at 37° C. (±0.5° C.).

FIG. 5 shows the human plasma isovaleramide concentration profiles following oral administration of 300 mg of isovaleramide in solution form or encapsulated isovaleramide immediate release pellets in the fasted and fed states.

FIG. 6 shows the human plasma isovaleramide concentration profiles following oral administration of 300 mg of encapsulated isovaleramide extended release pellets in the fasted state.

FIG. 7 shows the human plasma isovaleramide concentration profiles following oral administration of 600 mg of isovaleramide in extended release tablets in the fasted state.

### DETAILED DESCRIPTION OF THE INVENTION

Overview

Unless otherwise defined herein, scientific and technical terms used in connection with the present invention shall have the meanings that are commonly understood by those of ordinary skill in the art. Furthermore, unless otherwise required by context, singular terms shall include pluralities and plural terms shall include the singular. Generally, nomenclatures used in connection with, and standard techniques described herein are those well known and commonly used in the art. For example, in the framework of the present invention, the expressions “active pharmaceutical ingredient (API),” “active compound”, “active ingredient”, “drug” and “drug substance” are synonymous.

Developing extended release formulations of active compounds that exhibit characteristics such as a short biological half-life, a high daily dose, high water solubility and a temperature dependent instability at temperatures common to many of the processing steps used in the manufacture of pharmaceutical dosage forms, can pose a considerable challenge. However, there exists a need for developing such formulations in order to effectively treat a variety of pathological conditions such as CNS disorders.

In response to this need, the present invention provides extended release compositions of active compounds that pose such challenges. The formulations disclosed herein are designed to deliver a specific amount of drug over a specific course of time to achieve a therapeutic plasma drug concentration, while minimizing peak to trough differences that occur in vivo. This is achieved despite the fact that the drug is highly water soluble and exhibits temperature dependent instability.

“Extended release” as used herein, means the release of an active compound whereby the level of active compound available to the host is maintained at some level over a period of time. This is distinguished from “controlled release” which typically is broadly defined to include instantaneous release, delayed release and extended release concepts. “Instantaneous release” or “immediate release” refers to immediate release to the biosystem of the host while “delayed release” means the active ingredient is not made available to the host until some time delay following administration.

The water solubility of the drug substance as used herein, is defined according to its dose number, Do, for oral drug delivery. The dose number for a drug is defined by the ratio of its dose concentration to its saturation solubility in a reference fluid according to the following calculation:

$$Do = \frac{{M}_{o} / {V}_{o}}{{C}_{s}}$$

where: Mₒ is the dose of the drug (mg), Vₒ is the reference fluid volume (mL) and Cₛ is the saturation solubility of the drug in the reference fluid (mg/mL) at 37° C. Representative reference fluids for oral drug delivery are aqueous based solutions within the relevant physiologic pH range of the gastrointestinal tract (i.e., pH 1-pH 7.5). A representative reference fluid volume for oral drug delivery is 250 mL. A drug substance is considered to be highly soluble if at its highest dose the Do value is ≦1. For example, isovaleramide, a highly water soluble drug, is a neutral molecule that exhibits pH independent solubility over the physiologic pH range of the gastrointestinal tract. The solubility of isovaleramide in water at 37° C. is approximately 70 mg/mL. The upper range of a single daily dose of isovaleramide could be 1200 mg to 2400 mg. The Do number range for isovaleramide, calculated using a reference volume of 250 mL and the single daily dose range of 1200 mg to 2400 mg, is 0.07 to 0.14.

“Thermal instability” or “temperature dependent instability” as used herein, means unfavorable physical or chemical change in the active compound such as degradation, insolubility, sublimation and decomposition associated with rise in temperature during the formulation process.

Although the compositions of the present invention are designed to achieve extended release of the active compounds of the composition, instantaneous release and delayed release properties also can be designed into the instant compositions without departing from the spirit of the invention.

“Rate controlling polymer system” as used herein, refers to an excipient system in the pharmaceutical composition comprising one or more hydrophilic and/or hydrophobic polymers that controls the rate of water influx into and/or the release of drug substance from the composition when the pharmaceutical composition is exposed to an aqueous environment.

“Non-friable” as used herein, refers to an entity that cannot be easily crumpled, pulverized or reduced to powder when subjected to the mechanical stresses of the unit operations of the pharmaceutical manufacturing processes. “Fillers,” binders” and “binding agents” as used herein, refer to additives that can be mixed with the API in a suitable solvent so that upon subsequent drying, they render the inner core of the pellet non-friable.

“Granulation” as used herein, refers to the process of wet massing the drug substance with excipients such as fillers, binding agents, rate controlling polymers and the like using a suitable solvent.

“Extrusion” as used herein, refers to the process that forms the granulation into rod-shaped cylinders by passing the wet mass through dies.

“Spheronization” as used herein, refers to the process of transforming the rod-shaped extrudate into spheroids or rounded pellets using a spheronizer.

“Sealing coat” as used herein refers to a primary coating of the inner core of the pellet to facilitate the subsequent coating of the rate controlling polymer system or to enhance its rate controlling properties.

Temperature Dependent Instability Study

The type of temperature dependent instability of the active compound was studied in detail in order to determine the appropriate formulation methodology that could be suitable.

The temperature dependent physical instability of the drug substance was studied in detail in order to determine the appropriate pharmaceutical formulation processes and temperature ranges suitable for the preparation of the pharmaceutical compositions of isovaleramide. An automated thermogravimetric analysis (TGA) method was used to determine the relative rates of isovaleramide sublimation. Isothermal TGA was conducted at 30° C., 35° C., 40° C., 45° C., 50° C. and 60° C. for 24 hours. Samples were prepared by first taring a platinum TGA pan on the TGA system. A sample amount of isovaleramide of 10 mg-15 mg was then added to the pan and distributed evenly. The sample pan was then loaded onto the TGA autosampler for subsequent analysis. Samples were initially equilibrated at their respective isothermal temperature and then monitored for 24 hours. Data sampling was accomplished at a rate of 600 seconds/point. Sample replicates ranged from 1 to 3. TGA instrument and operating conditions were as follows:

| | |
|---|---|
| Instrument: | TGA Q500 (TA instruments) |
| Balance Purge Gas: | Nitrogen |
| Sample Purge Gas: | Nitrogen |
| Balance Purge Flow: | 40 mL/min |
| Sample Purge Flow: | 60 mL/min |

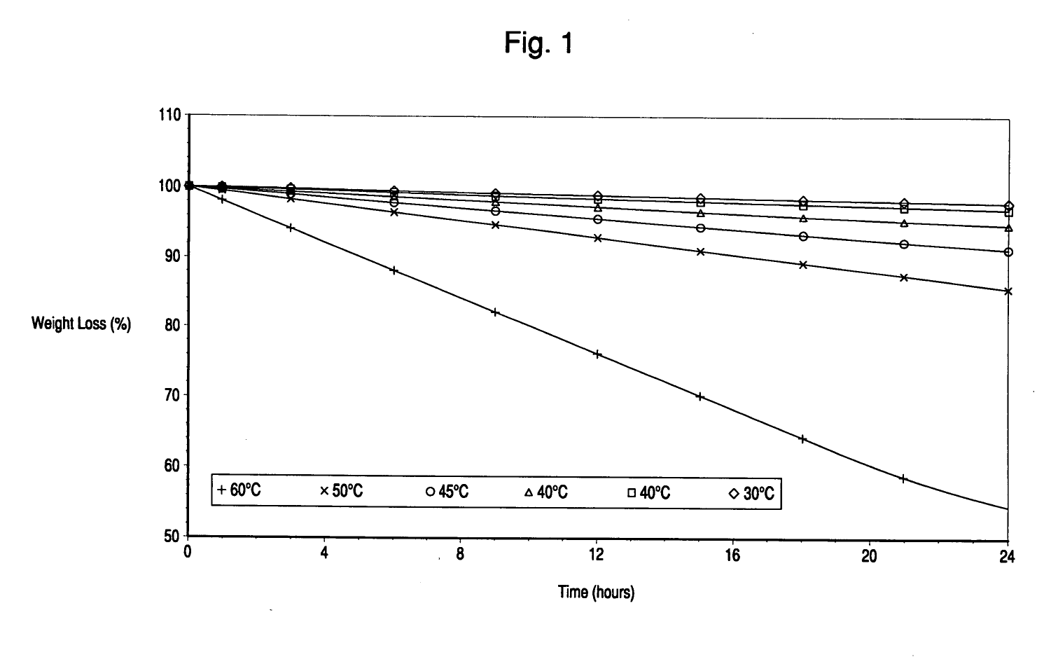

The results of the TGA evaluations are reported in Table 1 and FIG. 1.

TABLE 1
Thermogravimetric Analysis Results for Isovaleramide Bulk
Drug Substance at Various Temperatures

| Iso- | | | | | | | | | |
|---|---|---|---|---|---|---|---|---|---|
| thermal | | | | | | | | | |
| Con- | Reduction in Weight %^{a} | | | | | | | | |
| dition | 1 hr | 3 hr | 6 hr | 9 hr | 12 hr | 15 hr | 18 hr | 21 hr | 24 hr |
| 30° C. | 0.05 | 0.12 | 0.25 | 0.41 | 0.62 | 0.82 | 1.01 | 1.20 | 1.32 |
| 35° C. | 0.10 | 0.28 | 0.55 | 0.83 | 1.12 | 1.41 | 1.67 | 1.92 | 2.17 |
| 40° C. | 0.14 | 0.53 | 1.10 | 1.65 | 2.27 | 2.83 | 3.43 | 4.02 | 4.58 |
| 45° C. | 0.27 | 0.91 | 1.97 | 2.95 | 3.95 | 4.98 | 6.02 | 7.05 | 8.01 |
| 50° C. | 0.54 | 1.65 | 3.35 | 5.05 | 6.68 | 8.35 | 10.04 | 11.77 | 13.43 |
| 60° C. | 1.98 | 5.96 | 11.90 | 17.79 | 23.61 | 29.37 | 35.03 | 40.53 | 44.74 |

^{a}Values for 35° C., 40° C. and 60° C. are the mean of three replicates, all other temperatures are single runs. The standard deviations associated with the 35° C., 40° C. and 60° C. analyses were less than 0.12%, 0.43% and 1.93%, respectively.

Preparation of an Extended Release Formulation

With the present invention it was found that extended release compositions comprising pellet cores, such pellet cores comprising immediate release pellets that are coated with a release controlling polymeric membrane, provide prolonged in vivo isovaleramide exposure as compared to a non-disintegrating matrix tablet dosage form. Multiparticulate dosage forms can have a potential advantage over non-disintegrating matrix tablet dosage forms for extended release drug delivery when colonic drug absorption is integral to achieve the desired in vivo plasma concentration profile. Pellet formulations disperse as they transit in the gastrointestinal tract. In the colon this dispersive nature translates to a greater surface area exposure per drug loaded pellet, which is critical due to the lower water content in the colon, and a longer absorption time, that is a longer absorption window, relative to a non-disintegrating tablet dosage form.

Immediate Release Composition

By an “immediate release composition” it is meant an oral dosage form that is formulated to release substantially the entire active ingredient on administration with no delayed or extended release effect. Such a composition for the purposes of the present invention is, at least initially, in the form of a pellet (a term used interchangeably with “bead”, “beadlet” or inner core, herein). The immediate release pellet can also serve as a precursor to an extended or a delayed release pellet.

The non-active ingredients and processes for preparing such immediate release pellets are well known in the art; the present invention is not limited in this respect. See, for example, Remington's Pharmaceutical Sciences, 18^{th} Edition, A. Gennero, Ed., Mack Publishing Co. (Easton, Pa. 1990), Chapters 88-91, the entries of which are hereby incorporated by reference in their entirety.

For instance, an immediate release pellet can be prepared by mixing the isovaleramide with inactive pharmaceutical ingredients (also referred to as excipients) including diluents (or fillers), binding agents, disintegrants, glidants and lubricants. The amount of drug relative to total amount of the immediate release composition is preferably 50% (w/w) to 90% (w/w) but most preferred 80% (w/w) to 85% (w/w).

Bulking agents employable in these compositions may be chosen from, among others: microcrystalline cellulose, dicalcium phosphate, lactose, dextrose, sucrose, fructose, calcium sulfate and starch. Such bulking agents are typically present in the range of about 5% (w/w) to about 50% (w/w), with a preferred range of about 5% (w/w) to about 20% (w/w).

The immediate release composition may contain one or more binders to impart cohesiveness to the pellet formulation. Such binders are well known in the art, and include such substances as povidone, starch, gelatin, maltodextrin, methylcellulose, hydroxypropyl cellulose, hydroxypropyl methylcellulose, carboxymethylcellulose, sucrose solution, dextrose solution, acacia, tragacanth and locust bean gum, which may be applied wet. The binding agent may be present in the composition in an amount of from about 0.5% (w/w) to about 25% (w/w), preferably from about 5% (w/w) to about 15% (w/w).

The immediate release compositions can also contain one or more of the following excipients including disintegrants such as crosslinked sodium carboxymethylcellulose, sodium starch glycolate and crospovidone; glidants such as talc, starch and colloidal silicon dioxide; and lubricants such as talc, sodium stearyl fumarate, and the metallic stearates among others.

The immediate release pellet compositions can be made by, for example, granulation techniques such as wet granulation (low shear, high shear or fluid bed processor) or dry granulation, followed by sieving; extrusion and spheronization (marumerization); rotogranulation; roller compaction; or any agglomeration process that results in a pellet of reasonable robustness, drug load and range of size. For extrusion and spheronization, the preferred method, the drug and other additives are granulated by addition of a binder solution. The wet mass is passed through an extruder equipped with a certain size screen, and the extrudate is spheronized using a marumerizer. The resulting pellets are dried and sieved for further applications. Alternatively, the immediate release pellet compositions are prepared by solution or suspension layering, whereby a drug solution or dispersion, with or without a binder and optionally an anti-tacking agent such as talc, is sprayed onto an inert core or starting seed (either prepared or a commercially available product) in a fluid bed processor or other suitable equipment. The inert cores or starting seeds can be, for example, sugar spheres or spheres made from microcrystalline cellulose. The drug thus is coated on the surface of the starting seeds. The drug may also be layered onto the drug-containing immediate release pellet compositions described above, if desired. Following drug layering, the resulting drug-loaded pellets are dried for further applications.

A protective layer, or overcoat, such as a film coat, may be desired to ensure that the drug-loaded pellet compositions do not aggregate during further processing or upon storage. The protective coating layer may be applied to the surface of the pellet composition, either a drug-containing core or a drug-layered core, by conventional coating techniques such as pan coating or fluid bed processor coating using solutions of polymers in water or suitable organic solvents or by using aqueous polymer dispersions. OPADRY®, OPADRY II® (Colorcon) and corresponding color and colorless grades from Colorcon can be used to protect the pellets from being tacky and provide colors to the product. The suggested levels of protective or color coating are from about 1% to about 6%, preferably about 2% to about 3% (w/w).

Extended Release Pellet Composition

Isovaleramide extended release pellet compositions can be prepared, for example, by coating immediate release pellets with release controlling polymers. First, the immediate release pellet composition containing isovaleramide is prepared by a method described above. The immediate release pellet is then coated with a release controlling polymeric membrane. The release controlling polymer layer may be applied immediately to the surface of the pellet composition by conventional coating techniques, such as fluid bed processor coating, using solutions of polymers in water or suitable organic solvents or by using aqueous polymer dispersions. As an alternative embodiment, the release controlling membrane can separate additional drug layers on the core; for instance, after applying the release controlling polymer layer, another drug layer can be applied, which is followed by another release controlling layer, etc. Suitable rate controlling polymer materials include ammonio methacrylate copolymer, cellulose derivatives such as ethylcellulose, cellulose acetate, cellulose acetate butyrate, or polyvinyl acetate and the like and any copolymers or derivatives of the aforementioned. In addition, there may be provided, in certain embodiments, one or more plasticizers that are used in the rate controlling polymer formulation such as triethyl citrate, triacetin, dibutyl sebacate, diethyl phthalate, propylene glycol, polyethylene glycol and the like. The thickness of the coating affects the release profile, and so this parameter can be used to customize the profile. The suggested coating levels are from about 1% to about 40% (w/w), preferably about 5% to about 35% (w/w)

Extended Release Tablet Composition

Isovaleramide extended release tablet compositions can be prepared, for example, by dry blending the tablet formulation components followed by tablet compression (direct compression) or alternatively a wet granulation process is employed to produce granules comprising isovaleramide and select formulation excipients followed by the preparation of a dry blend of the isovaleramide containing granules with additional formulation excipients and tablet compression.

The extended release rate for isovaleramide is achieved based on the nature and usage level (or concentration) of the release controlling polymer used in the tablet formulation such as polymers exhibiting pH independent or pH dependent swelling (enteric polymers) used alone or in combination with one another. Suitable polymers exhibiting pH independent swelling that may be included in the extended release composition, but are not limited to, hydroxypropyl methylcellulose, hydroxypropyl cellulose, hydroxyethyl cellulose, methylcellulose, polyethylene oxide, polyvinyl alcohol, and xanthan gum. A particularly preferred hydrophilic polymer exhibiting pH independent swelling is hydroxypropyl methylcellulose. Suitable polymers exhibiting pH dependent swelling that may be included in the extended release composition, but are not limited to, Carbomer 941, NF (Carbopol 971P, NF), Carbomer 934P, NF (Carbopol 974P, NF), acrylic acid copolymers and guar gum. A particularly preferred hydrophilic polymer exhibiting pH dependent swelling include the carbomers. The suggested levels of the suitable release controlling polymers are 5% to about 40% (w/w), preferably 15% to 30% (w/w).

Additionally, water insoluble, low permeability polymers or hydrophobic matrix former can be included in the composition to function as release controlling excipients. The water insoluble, low permeability polymers or hydrophobic matrix formers that may be included in the extended release composition, but are not limited to, ethylcellulose, cellulose acetate, ammonio methacrylate copolymer (EUDRAGIT® RS PO), glyceryl palmitostearate, glyceryl behenate, glyceryl monostearate and other such waxes and waxy substances.

The extended release tablet composition may also contain bulking agents (tablet diluents) chosen from, among others: microcrystalline cellulose, dicalcium phosphate, lactose, dextrose, sucrose, fructose, calcium sulfate and starch. Such bulking agents are typically present in the range of about 5% (w/w) to about 50% (w/w), with a preferred range of about 5% (w/w) to about 20% (w/w).

The extended release tablet composition may contain one or more binders to impart cohesiveness to the compressed tablet formulation. Such binders are well known in the art, and include such substances as povidone, hydroxypropyl cellulose, hydroxypropyl methylcellulose, methylcellulose, starch, carboxymethylcellulose, gelatin, maltodextrin, sucrose solution, dextrose solution, acacia, tragacanth and locust bean gum, which may be applied wet. The binding agent may be present in the composition in an amount of from about 0.25% (w/w) to about 10%(w/w), preferably from about 0.3% (w/w) to about 5% (w/w).

The extended release tablet compositions can also contain one or more of the following excipients including glidants such as talc, starch and colloidal silicon dioxide; and lubricants such as talc, sodium stearyl fumarate, and the metallic stearates, among others.

### EXAMPLES

In order that this invention may be better understood, the following examples are set forth. These examples are for the purpose of illustration only and are not to be construed as limiting the scope of the invention in any manner. Numerous other variations of the present invention will be appreciated by those skilled in the art, in view of the disclosure herein. The exact compositions, methods of preparation and embodiments shown are not limiting of the invention, and any obvious modifications will be apparent to one skilled in the art.

### Example 1

### Isovaleramide Immediate Release Pellets

Isovaleramide immediate release pellets were manufactured by an extrusion and spheronization process. The batch formula for the immediate release pellets is provided in Table 2. A granulation consisting of isovaleramide, hydroxypropyl methylcellulose, and microcrystalline cellulose was produced using an aqueous high shear granulation process and a Glatt-Powrex vertical granulator (Model FM-VG 65M/25/10). The granulation was extruded using a dome granulator (LCI-Fuji Paudal, Model DG-L2), and spheronized using a marumerizer (LCI-Fuji Paudal, Model QJ-400G). The spheronized product was dried using a fluid bed processor unit (Glatt Powder Coater Granulator, Model GPCG-15) and then screened (18 mesh/40 mesh sieves). The target process parameters values for the stages of manufacture for the isovaleramide immediate release pellets are provided in Table 3. The immediate release composition produced contained about 85% (w/w) isovaleramide.

TABLE 2
Batch Formula for Isovaleramide Immediate Release Pellets

| Component | Amount (kg) |
|---|---|
| Isovaleramide | 3.825 |
| Hypromellose (hydroxypropyl | 0.225 |
| methylcellulose, USP | |
| (Methocel E5 Premium LV, Dow) | |
| Microcrystalline Cellulose, NF | 0.450 |
| (Avicel ® PH 302, FMC) | |
| Purified Water, USP^{a} | 0.810 |
| Total | 4.500 |

^{a}Removed during processing.

TABLE 3
Target Process Parameters Values for the Stages of Manufacture for
Isovaleramide Immediate Release Pellets

| Process | Parameter (Target Value)^{a} |
|---|---|
| Granulation | Blade speed (250 RPM) |
| | Chopper blade speed (1000 RPM) |
| | Binder solvent addition rate (40 g/min) |
| Extrusion | Feed screw rate (setting 0.6) |
| Spheronization | Plate speed (setting 0.6) |
| Drying | Fluid bed process air volume (250 CFM) |
| | Fluid bed inlet air temperature (45° C.) |
| | Fluid bed product temperature (35° C.) |
| | Fluid bed exhaust air temperature (35° C.) |

Addition batch formula for isovaleramide immediate release pellet compositions are provided in Table 4.

TABLE 4
Batch Formula for Additional Isovaleramide Immediate Release Pellets

| | Composition Number | | | |
|---|---|---|---|---|
| Component | 1 | 2 | 3 | 4 |
| Isovaleramide | 60 | 70 | 80 | 80 |
| Hypromellose (hydroxypropyl | 5 | 5 | 3 | 5 |
| methylcellulose), USP | | | | |
| Lactose Monohydrate, USP | 15 | 10 | 7 | NA |
| Microcrystalline Cellulose, NF | 20 | 15 | 10 | 15 |
| Water | ^{a} | ^{a} | ^{a} | ^{a} |
| Total | | | | |

^{a}Removed during processing.

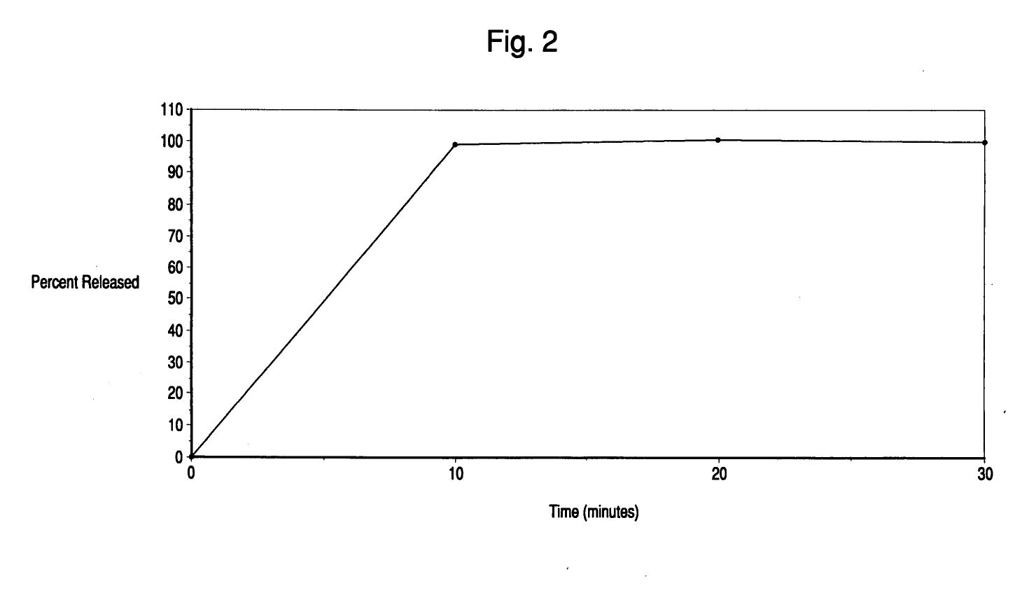

Average isovaleramide content and moisture values for representative lots of immediate release pellets are presented in Table 5. A representative dissolution profile for the immediate release composition (Table 2) is provided FIG. 2. Complete dissolution of the isovaleramide occurs within 10 minutes.

TABLE 5
Average Content and Moisture Values for Isovaleramide Immediate
Release Pellets

| Batch Number | Average Content (% LC)^{a} | Moisture (%)^{b} |
|---|---|---|
| PD0209-124 | 100.4 | 1.6 |
| PD0209-127 | 98.5 | 3.0 |
| PD0209-135 | 98.0 | 3.5 |
| PD0209-180 | 97.8 | 3.2 |
| PD0209-186 | 98.0 | 2.7 |
| PD0209-189 | 96.2 | 4.3 |
| B04046 | 96.4 | 3.8 |
| B03060 | 96.3 | 3.6 |
| B03061 | 96.4 | 3.8 |
| B04019 | 100.1 | 1.0 |

^{a}Determined by HPLC, LC = label claim
^{b}Determined by Karl Fischer titration

### Example 2

### Isovaleramide Extended Release Pellets

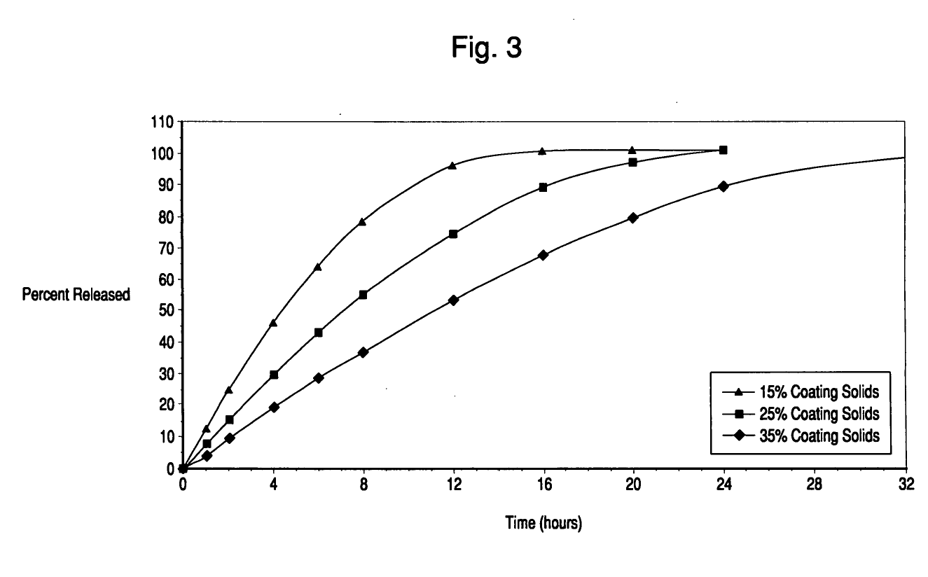

Isovaleramide extended release pellets (target batch size range 4.2 kg-5.5 kg) were manufactured by coating isovaleramide immediate release pellets with SURELEASE® Clear E-7-19010 coating dispersion (Colorcon, West Point, Pa.) using a fluid bed processor (Glatt Powder Coater Granulator, Model GPCG-15). SURELEASE Clear E-7-19010 is an ethylcellulose based aqueous dispersion having a target solids content of 25% (w/w). The SURELEASE® Clear coating dispersion was prepared by adding water to the dispersion to achieve a 15% (w/w) dispersion solids level and mixing for 20 minutes. The resulting 15% (w/w) dispersion was stirred throughout the coating process to prevent settling of coating components. Various coating levels of the 15% (w/w) dispersion were examined with the objective of achieving extended release pellets with different drug release rates. The target process parameters values for the extended pellet coating process are provided Table 6. Following the application of the 15% (w/w) dispersion the pellets were screened (18 mesh/40 mesh sieves) mixed with talc and then cured at 40° C. for approximately 24 hours. Following oven curing, the extended release pellet compositions were filled into size 00EL hard gelatin capsules using a MG2 Futura encapsulator. The compositions of isovaleramide XR pellets filled capsules are provided in Table 7. The average isovaleramide content and moisture values for representative lots of encapsulated extended release pellets are presented in Table 8. FIG. 3 shows the in vitro dissolution profiles for encapsulated ethylcellulose coated isovaleramide extended release pellets. The in vitro dissolution profiles show extended drug release, as defined by the time to release about 90% (t₉₀) of the label amount of isovaleramide in the encapsulated product, in about 10 hours (MXR1—15% coating solids), 16 hours (MXR-2—25% coating solids) and 23 hours (MXR-3—35% coating solids).

TABLE 6
Target SURELEASE ® Clear Coating Process Parameters Values

| Process | Parameter | Target Value |
|---|---|---|
| XR Pellet | Fluid bed process air volume | 250 CFM |
| Coating | Fluid bed inlet air temperature, | 60° C. |
| | Fluid bed product temperature, | 40° C. |
| | Fluid bed exhaust air temperature, | 35° C.-40° C. |
| | Spray rate | 38 g/min-45 g/min |

TABLE 7
Components Amounts Per Dosage Unit in
Isovaleramide Extended Release Capsules

| | MXR-1 | MXR-2 | MXR-3 |
|---|---|---|---|
| | Amount, | Amount, | Amount, |
| | mg/capsule | mg/capsule | mg/capsule |
| Component | (% w/w) | (% w/w) | (% w/w) |
| Isovaleramide | 300.00 | 300.00 | 300.00 |
| | (54.18%) | (49.17%) | (43.86%) |
| Hypromellose (hydroxypropyl | 17.65 | 17.65 | 17.65 |
| methylcellulose), USP | (3.19%) | (2.89%) | (2.58%) |
| (Methocel E5 Premium LV, Dow) | | | |
| Microcrystalline Cellulose, NF | 35.29 | 35.29 | 35.29 |
| (Avicel ® PH 302, FMC) | (6.37%) | (5.78%) | (5.16%) |
| Ethylcellulose-based Coating Dispersion | 62.28 | 117.65 | 190.05 |
| (SURELEASE ® Clear, | (11.25%) | (19.28%) | (27.78%) |
| E-7-19010 Colorcon)^{a} | | | |
| Talc, USP | 8.47 | 9.60 | 11.08 |
| (Altalc ® 300 V, Luzenac) | (1.53%) | (1.57%) | (1.62%) |
| Purified Water, USP | ^{b} | ^{b} | ^{b} |
| Hard Gelatin Capsule, size 00EL white | 130.00 | 130.00 | 130.00 |
| opaque, CAPSUGEL ® | (23.48%) | (21.30%) | (19.00%) |
| TOTAL | 553.69 | 610.19 | 684.07 |
| | (100%) | (100%) | (100%) |

^{a}Amount reported as dispersion solids based on the dispersion solids content of 25% (w/w).
^{b}Removed during processing

TABLE 8
Average Content and Moisture Values for Encapsulated Isovaleramide
Extended Release Pellets

| | Average Content | |
|---|---|---|
| Batch Number | (% LC)^{a} | Moisture (%)^{b} |
| PD0209-154 (MXR-15) | 96.9 | 0.5 |
| PD0209-155 (MXR-22.5) | 98.4 | 0.5 |
| PD0209-156 (MXR-30) | 98.6 | 0.5 |
| PD0209-181 (MXR-15) | 100.5 | ND |
| PD0209-187 (MXR-25) | 97.1 | ND |
| PD0209-190 (MXR-35) | 97.0 | ND |
| B04054 (MXR-15) | 98.7 | 0.3 |
| B03055a (MXR-25) | 100.8 | 0.3 |
| B03056 (MXR-35) | 100.7 | 0.5 |

ND = not determined
^{a}Determined by HPLC. LC = label claim
^{b}Determined by Karl Fischer titration

### Example 3

### Isovaleramide Extended Release Tablets

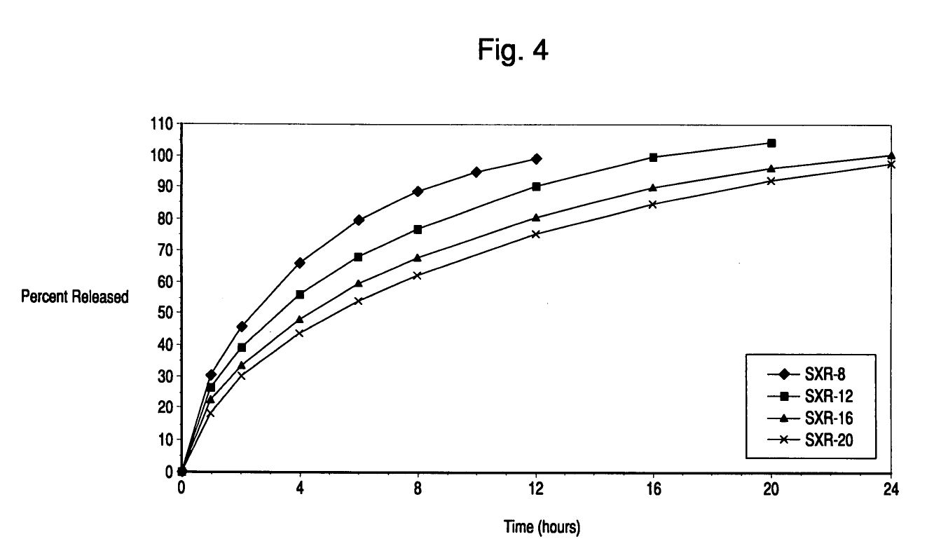

A series of extended release tablets were formulated to contain a dose equivalent to 600 mg isovaleramide (Table 9). The manufacture of the isovaleramide extended release tablets was initiated with the production of an intermediate granulation using a high shear wet granulation process (target batch size range 4.8 kg-5.6 kg). The isovaleramide, hydroxypropyl methylcellulose and colloidal silicon dioxide components of the granulation were dry blended in a low shear diffusional mixer (e.g., Patterson-Kelly V-blender, 16 qt shell) preparing a pre-blend. The pre-blend of isovaleramide, hydroxypropyl methylcellulose and colloidal silicon dioxide was then granulated by a high shear granulation process using a granulation solution comprising povidone, alcohol and water and a Glatt-Powrex vertical granulator (Model FM-VG 65M/25/10). The final granulated product was oven dried at 35° C. for approximately 24 hours to a moisture content level of not more than 2.0%. The dried granules were screened (18 mesh sieve) and then dry blended (5.5 kg-6.5 kg batch size range) in a low shear diffusional mixer (Patterson-Kelly V-blender, 16 qt shell) with microcrystalline cellulose (SXR-8 and SXR-12, only), colloidal silicon dioxide, talc and sodium stearyl fumarate to produce a final blend for tableting. The final blends are tableted on a rotary tablet press (Kilian, Model S250-ZS) at a target tablet weight range to provide a dose equivalent to 600 mg isovaleramide and then dedusted. The resultant bulk core tablet formulations were film coated (4.5 kg-5.2 kg batch size range) in a coating pan (O'Hara LabCoat II-X, 19 inch pan, one spray gun) using an aqueous based hydroxypropyl methylcellulose coating system. The target process parameters values for the stages of manufacture for the isovaleramide extended release tablets are provided in Table 10. Average isovaleramide content and moisture values for representative lots of extended release tablets are presented in Table 11. FIG. 4 shows the in vitro dissolution profiles for a series of isovaleramide extended release tablets. The in vitro dissolution profiles show extended drug release, as defined by the time to release about 90% (t₉₀) of the label amount of isovaleramide in the encapsulated product, in about 8 hours (SXR-8), 12 hours (SXR-12), 16-hours (SXR-16) and 20 hours (SXR-20).

TABLE 9
Components Amounts Per Dosage Unit in Isovaleramide Extended Release
Tablets

| | SXR-8 | SXR-12 | SXR-16 | SXR-20 |
|---|---|---|---|---|
| | Amount, | Amount, | Amount, | Amount, |
| | mg/tablet | mg/tablet | mg/tablet | mg/tablet |
| Component | (% w/w) | (% w/w) | (% w/w) | (% w/w) |
| Isovaleramide | 600.0 | 600.0 | 600.0 | 600.0 |
| | (58.78%) | (58.78%) | (58.49%) | (58.79%) |
| Hypromellose (hydroxypropyl | NA | 150.0 | 300.0 | 250.0 |
| methylcellulose), USP | | (14.70%) | (29.25%) | (24.49%) |
| (Methocel K100M Premium CR, Dow) | | | | |
| Hypromellose (hydroxypropyl | 150.0 | NA | NA | NA |
| methylcellulose), USP | (14.70%) | | | |
| (Methocel K(100 LV Premium CR, Dow) | | | | |
| Hypromellose (hydroxypropyl | 49.5 | NA | NA | NA |
| methylcellulose), USP | (4.85%) | | | |
| (Methocel K4M Premium CR, Dow) | | | | |
| Carbomer 941, NF | NA | NA | NA | 50.0 |
| (Carbopol 971P) | (4.90) | | | |
| Microcrystalline Cellulose, NF | 100.5 | 150.0 | NA | NA |
| (Avicel ® PH 302, FMC) | (9.85%) | (14.70%) | | |
| Povidone K(25, USP | 31.5 | 31.5 | 36.0 | 31.5 |
| (Kollidon 25, BASF) | (3.09%) | (3.09%) | (3.51%) | (3.08%) |
| Povidone K(90, USP | 3.5 | 3.5 | 4.0 | 3.5 |
| (Kollidon 90F, BASF) | (0.343%) | (0.343%) | (0.390%) | (0.340%) |
| Colloidal Silicon Dioxide, NF | 25.1 | 25.1 | 25.0 | 25.1 |
| (CAB-O-SIL MSP, Cabot) | (2.46%) | (2.46%) | (2.44%) | (2.44%) |
| Talc, USP | 20.0 | 20.0 | 20.0 | 20.0 |
| (Talc Imperial 1889L, Luzenac) | (1.96%) | (1.96%) | (1.95%) | (1.95%) |
| Sodium Stearyl Fumarate, NF | 10.0 | 10.0 | 10.0 | 10.0 |
| (Pruv, JRS Pharma) | (0.98%) | (0.98%) | (0.97%) | (0.97%) |
| Film Coating System | 30.6 | 30.6 | 30.8 | 30.6 |
| (Opadry II White, 33G28523, Colorcon) | (3.00%) | (3.00%) | (3.00%) | (3.00%) |
| Dehydrated Alcohol, USP | ^{a} | ^{a} | ^{a} | ^{a} |
| Purified Water, USP | ^{a} | ^{a} | ^{a} | ^{a} |
| TOTAL | 1020.7 | 1020.7 | 1025.8 | 1020.7 |
| | (100%) | (100%) | (100%) | (100%) |

NA = not applicable
^{a}Removed during processing

TABLE 10
Target Process Parameter Values for the Stages Of Manufacture for
Isovaleramide Extended Release Tablets

| Stage | Process | Parameter | Target Value (Range)^{a} |
|---|---|---|---|
| Isovaleramide | Dry Blending | Blending times | |
| Granules | | Blending procedure 1 | 6 minutes (±½ minutes) |
| Manufacture | | Blending procedure 2 | 5 minutes (±½ minutes) |
| | Granulation | Main blade speed | 250 RPM (±25 RPM) |
| | | Chopper blade speed | 1000 RPM (±100 RPM) |
| | | Binder solvent addition rate | 60 g/min (45-75 g/min) |
| | Drying | Temperature | 35° C. (30-45° C.) |
| Isovaleramide SXR | Dry Blending | Blending times | |
| Tablet Manufacture | | All blending procedures | 5 minutes (±½ minutes) |
| (bulk core tablets) | Tableting | Pre-compression setting | 3 mm-3.5 mm |
| | | Main compression setting | 3.8 mm 4 mm |
| | | Fill depth | 14.25 mm-18.6 mm |
| | | Fill shoe speed | 75 RPM-90 RPM |
| | | Press speed | 26 RPM |
| | | Deduster frequency | 45.7 kHz |
| | | Deduster power parameter | 8.5 Pulse width |
| Isovaleramide SXR | Film Coating | Inlet temperature | 60° C. (55-70° C.) |
| Tablet Manufacture | | Exhaust temperature | 42° C. (35-50° C.) |
| (bulk film coated | | Air flow | 190 CFM (180-210 |
| tablets) | | | CFM) |
| | | Atomization Air | 30 PSI (28-32 PSI) |
| | | Pattern air | 35 PSI (33-37 PSI) |
| | | Spray rate | 25 g/min (20-30 g/min) |
| | | Pan rotation | 8 RPM (7-9 RPM) |
| | | Pan differential pressure | −0.1 inches of H₂O C |
| | | | (−0.2 to −0.05 inches of |
| | | | H₂O C) |

TABLE 11
Average Content and Moisture Values for
Isovaleramide Extended Release Tablets

| Batch Number | Average Content (% LC)^{a} | Moisture (%)^{b} |
|---|---|---|
| PD0256-131 (SXR-8) | 102.9 | 1.3 |
| PD0256-080 (SXR-12) | 99.1 | 2.1 |
| PD0256-082 (SXR-16) | 99.0 | 2.3 |
| PD0256-081 (SXR-20) | 97.9 | 2.3 |
| B04049 (SXR-8) | 102.8 | 1.0 |
| B04047 (SXR-12) | 101.8 | 1.0 |
| B04048 (SXR-16) | 100.3 | 0.9 |

^{a}Determined by HPLC, LC = label claim
^{b}Determined by Karl Fischer titration

### Example 4

### Human Pharmacokinetic Evaluation of Isovaleramide Immediate Release Pellets

A single dose, randomized, crossover pharmacokinetic study was performed in humans (n=24 healthy subjects) to compare the pharmacokinetic profile of encapsulated isovaleramide immediate release pellets (300 mg dose strength) given in the fasted and fed states to that of an isovaleramide oral solution (300 mg dose strength). The mean pharmacokinetic parameters are listed in Table 12.

TABLE 12
Mean Human Pharmacokinetic Parameters Comparing 300 mg Dose
Strength Isovaleramide Immediate Release Pellets Dosed in the Fasted
(MIR-Fasted) and Fed States (MIR-Fed) to a 300 mg Dose Strength Oral
Solution

| Mean PK Parameter^{a} | Oral Solution | MIR-Fasted | MIR-Fed |
|---|---|---|---|
| Tₘₐₓ (hr) | 0.75 | 1.0 | 2.5 |
| Cₘₐₓ (μg/mL) | 4.62 | 4.91 | 3.66 |
| AUCₗₐₛₜ (hr · μg/mL)^{b} | 22.8 | 24.0 | 21.1 |
| Relative BA (%)^{c} | NA | 105 | 92.5 |

BA = bioavailability
^{a}Mean of 24 subjects.
^{b}AUC = area under the plasma concentration time profile calculated using the trapezoidal rule to the last sampling time point (36 hours)
^{c}Relative bioavailability calculated using the 300 mg oral solution dose as the reference formulation.

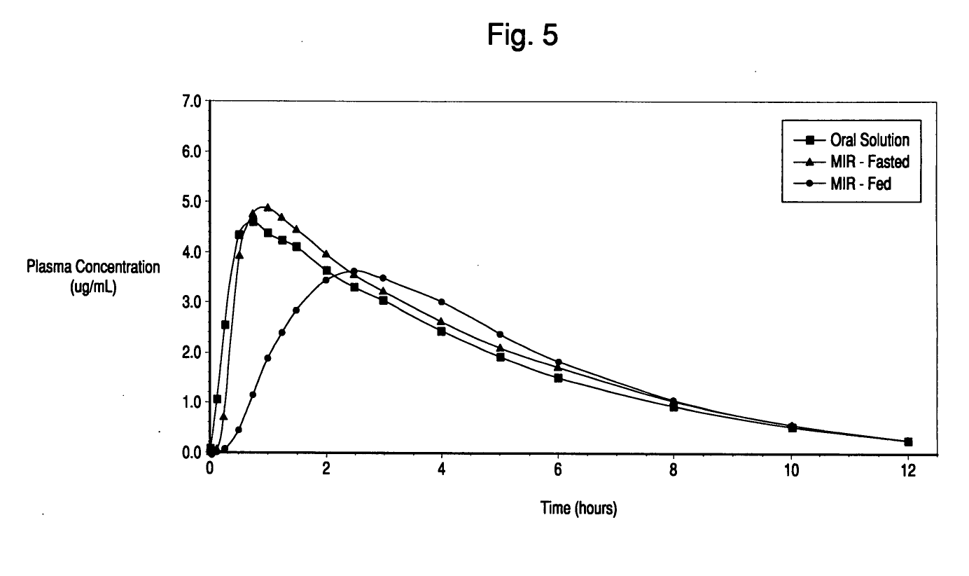

The mean Tₘₐₓ and Cₘₐₓ values in Table 12 are observed values from the mean isovaleramide plasma concentration versus time profiles (FIG. 5.) The AUCₗₐₛₜ values were calculated using the pharmacokinetic modeling software WinNonlin (Version 4.1, Pharsight Corporation, California). Relative bioavailability values were calculated as follows:

$$Relative BA ( \% ) = \frac{{AUC}_{last \text{-} MIR Formulation} \times {Dose}_{Oral Solution}}{{AUC}_{last \text{-} Oral Solution} \times {Dose}_{MIR Formulation}} \times 100 \%$$

### Example 5

### Human Pharmacokinetic Evaluation of Encapsulated Isovaleramide Extended Release Pellets

A single dose, randomized, crossover pharmacokinetic study was performed in humans (n=21 healthy subjects) to compare the pharmacokinetic profile of encapsulated isovaleramide extended release pellets (300 mg dose strength) given in the fasted state to that of an isovaleramide oral solution (300 mg dose strength). The mean pharmacokinetic parameters are listed in Table 13.

TABLE 13
Mean Human Pharmacokinetic Parameters Comparing 300 mg Dose
Strength Isovaleramide Extended Release Pellets Dosed in the Fasted State

| | Oral | | | |
|---|---|---|---|---|
| Mean PK Parameter^{a} | Solution | MXR-15 | MXR-25 | MXR-35 |
| Tₘₐₓ (hr) | 1.0 | 3.0 | 5.0 | 6.0 |
| Cₘₐₓ (μg/mL) | 4.49 | 0.78 | 0.59 | 0.46 |
| AUC_{inf} (hr · μg/mL)^{b} | 23.8 | 17.7 | 15.0 | 13.9 |
| Relative BA (%)^{c} | NA | 74.4 | 63.1 | 58.43 |

BA = bioavailability
^{a}Mean of 24 subjects.
^{b}AUC = area under the plasma concentration time profile estimated to infinity. Estimate values determined using pharmacokinetic modeling software WinNonlin (Version 4.1 Pharsight Corporation, California).
^{c}Relative bioavailability calculated using the 300 mg oral solution dose as the reference formulation.

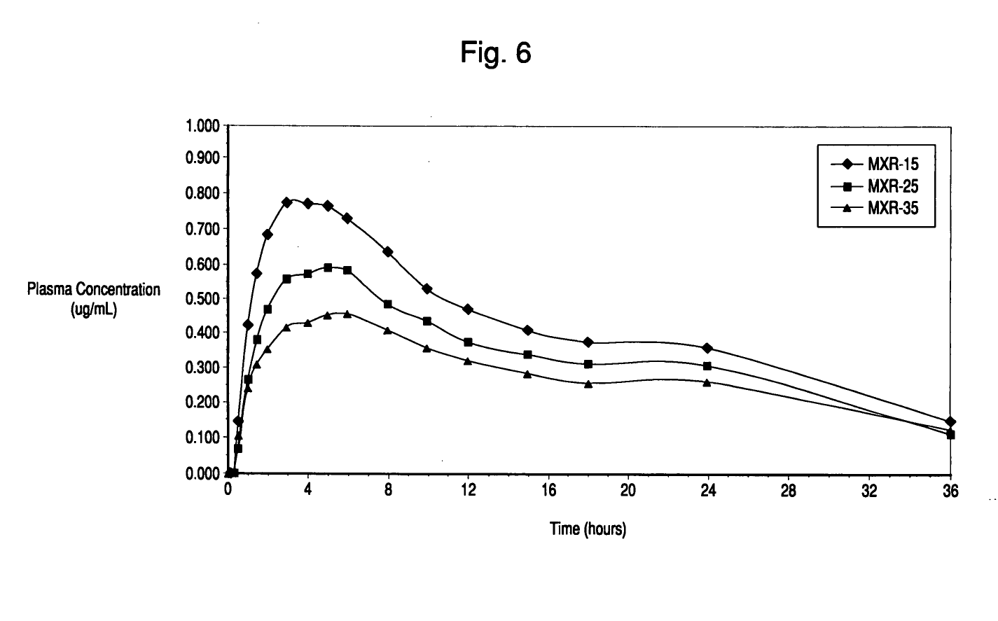

The mean Tₘₐₓ and Cₘₐₓ values in Table 13 are observed values from the mean isovaleramide plasma concentration versus time profiles (FIG. 6.) The AUC_{inf} values were estimated using the pharmacokinetic modeling software WinNonlin (Version 4.1, Pharsight Corporation, California). Relative bioavailability values were calculated as follows:

$$Relative BA ( \% ) = \frac{{AUC}_{inf \text{-} MXR Formulation} \times {Dose}_{Oral Solution}}{{AUC}_{inf \text{-} Oral Solution} \times {Dose}_{MXR Formulation}} \times 100 \%$$

### Example 6

### Human Pharmacokinetic Evaluation of Isovaleramide Extended Release Tablets

A single dose, randomized, crossover pharmacokinetic study was performed in humans (n=24 healthy subjects) to evaluate the pharmacokinetic profile of isovaleramide extended release tablets (600 mg dose strength) given in the fasted state. The mean pharmacokinetic parameters are listed in Table 14.

TABLE 14
Mean Human Pharmacokinetic Parameters Comparing 600 mg Dose
Strength Isovaleramide Extended Release Tablets Dosed in the Fasted
State

| Mean PK Parameter^{a} | SXR-8 | SXR-12 | SXR-16 |
|---|---|---|---|
| Tₘₐₓ (hr) | 3.0 | 3.0 | 3.0 |
| Cₘₐₓ (μg/mL) | 4.16 | 2.85 | 2.43 |
| AUC_{inf} (hr · μg/mL)^{b} | 38.8 | 36.4 | 30.43 |
| Relative BA (%)^{c} | 81.5 | 76.5 | 63.9 |

BA = bioavailability
^{a}Mean of 24 subjects.
^{b}Area under the plasma concentration-time profile estimated to infinity.
^{c}Relative bioavailability calculated using a 300 mg oral solution dose as the reference formulation. Oral solution pharmacokinetic data used for these calculations was obtained from Table 13.

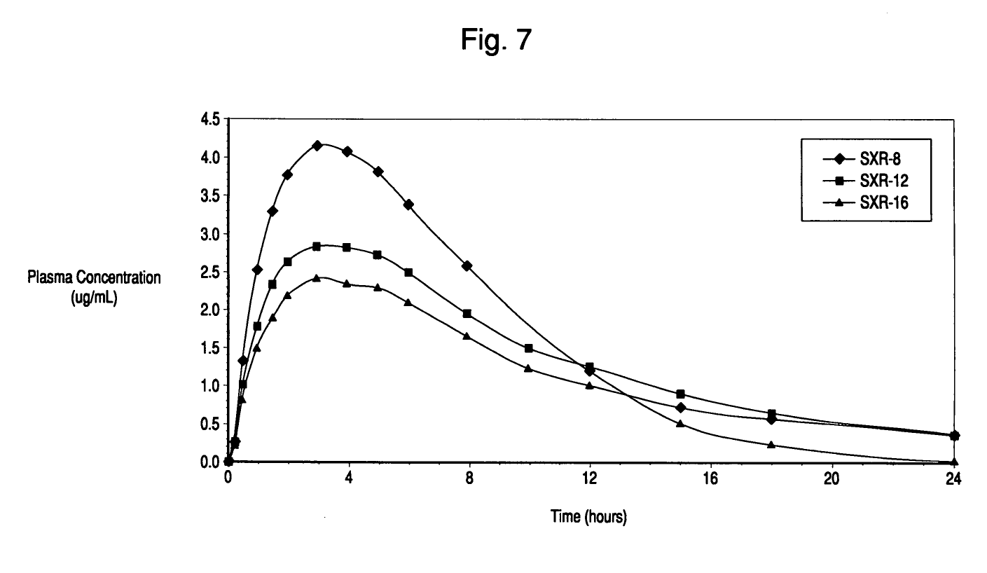

The mean Tₘₐₓ and Cₘₐₓ values in Table 14 are observed values from the mean isovaleramide plasma concentration versus time profiles (FIG. 7). The AUC_{inf} values were estimated using the pharmacokinetic modeling software WinNonlin (Version 4.1, Pharsight Corporation, California). Relative bioavailability values were calculated as follows (using the 300 mg oral solution pharmacokinetic data provided in Table 13):

$$Relative BA ( \% ) = \frac{{AUC}_{inf \text{-} SXR Formulation} \times {Dose}_{Oral Solution}}{{AUC}_{inf \text{-} Oral Solution} \times {Dose}_{SXR Formulation}} \times 100 \%$$

We claim:

1. A pharmaceutical composition comprising an active pharmaceutical ingredient (API) formulated as a unitary body suitable for oral administration to a subject, wherein said API exhibits a property selected from the group consisting of highly water soluble, temperature-dependent instability and combination thereof, and wherein upon administration said composition provides a therapeutically effective blood plasma level of said API in said subject when dosed once or twice a day.

2. A pharmaceutical composition according to claim 1, wherein said subject is a mammal.

3. A pharmaceutical composition according to claim 1, wherein said subject is a human.

4. The composition according to claim 1, wherein said temperature-dependent instability comprises physical instability.

5. The composition according to claim 1, wherein said temperature dependent instability comprises chemical instability.

6. The composition according to claim 4, wherein said physical instability is sublimation.

7. The composition according to claim 5, wherein said chemical instability is decomposition.

8. The composition according to claim 1, wherein said unitary body is a capsule or a tablet.

9. The composition according to claim 1, wherein said unitary body comprises said API in the range of approximately 10% (w/w) to approximately 90% (w/w).

10. The composition according to claim 8, wherein said tablet comprises a substantially homogeneous mixture of approximately 10% (w/w) to approximately 60% (w/w) API, a rate controlling polymer system, and one or more pharmaceutically acceptable excipients.

11. The composition according to claim 10, wherein said tablet comprises said API in the range of approximately 55% (w/w) to approximately 60% (w/w).

12. The composition according to claim 8, wherein said capsule comprises multiparticulate pellets comprising an inner core coated with a substantially uniform layer of a rate controlling polymer system, wherein said inner core comprises a substantially homogeneous mixture of approximately 50% (w/w) to approximately 90% (w/w) API and one or more pharmaceutically acceptable excipients.

13. The composition according to claim 12, wherein said inner core comprises a substantially homogeneous mixture of approximately 80% (w/w) to approximately 85% (w/w) API.

14. The composition according to claim 12, optionally comprising a sealing coating between said inner core and said rate controlling polymer system.

15. The composition according to claim 12, wherein said pellets range in size from approximately 250 μm to approximately 1000 μm.

16. The composition according to claim 12, wherein said pellets range in size from about 425 μm to about 1000 μm.

17. The composition according to any one of the preceding claims, wherein said API is isovaleramide.

18. A process for preparing the composition of claim 1 comprising the following sequential steps:
(i) granulating said API with a component selected from the group consisting of a pharmaceutically acceptable filler, a binder and combinations thereof to form a mixture;
(ii) extruding and spheronizing said mixture to form inner core pellets;
(iii) coating said inner core pellets with a substantially uniform layer of a rate controlling polymer system; and
(iv) drying the resulting coated pellets.

19. The process according to claim 18, wherein the particle size of said inner core pellets is between about 250 μm and about 1000 μm.

20. The process according to claim 18, wherein the particle size of each of said inner core pellets is between about 425 μm and about 1000 μm.

21. The process according to claim 18, wherein said rate controlling polymer system is selected from the group consisting of ammonio methacrylate copolymer, cellulose derivatives, polyvinyl acetate, and derivatives thereof.

22. The process according to claim 21, wherein said cellulose derivatives are selected from a group consisting of cellulose acetate, cellulose acetate butyrate, hydroxypropyl methylcellulose, hydroxypropyl cellulose and ethylcellulose.

23. The process according to claim 21, wherein said rate controlling polymer system is ethylcellulose.

24. The process according to claim 18, wherein said coated multiparticulate pellet is optionally further coated with an additional layer of polymer.

25. The process according to claim 24, wherein said additional polymer is hypromellose.

26. The process according to claim 18, wherein said granulation is high shear granulation.

27. The process according to claim 18, wherein said drying comprises fluid bed drying or oven drying.

28. A process for preparing the composition according to claim 1 comprising the following sequential steps:
(i) mixing said API with excipients comprising at least one rate controlling polymer to form a substantially homogeneous extended release mixture;
(ii) compressing said mixture into tablets; and
(iii) optionally film-coating said tablets with excipients to make them suitable for oral administration.

29. The process according to claim 28, wherein said compression is direct compression.

30. A process for preparing the composition according to claim 1 comprising the following sequential steps:
(i) mixing said API with excipients comprising at least one matrix forming polymer to form a substantially homogeneous extended release mixture;
(ii) granulating said mixture to form extended release granules;
(iii) compressing said granules into tablets; and
(iv) optionally film-coating said tablets with excipients to make them suitable for oral administration.

31. The process according to claim 30, wherein said granulation is high shear granulation.

32. The process according to claims 28 or 30, wherein said excipients are selected from the group consisting of hydroxypropyl methylcellulose, ethylcellulose, hydroxypropyl cellulose, poly (ethylene oxide), polyvinyl alcohol, polyethylene glycol, titanium oxide, xanthan gum, carbomer, microcrystalline cellulose, lactose monohydrate, polyvinylpyrrolidone, colloidal silicon dioxide, glyceryl behenate, talc, sodium stearyl fumarate, magnesium stearate and combinations thereof.

33. The process according to claims 28 or 30, wherein said API is isovaleramide.
